(19) United States
(12) Patent Application Publication
(10) Pub. No.: US 2011/0152825 A1
(43) Pub. Date: Jun. 23, 2011

(54) PRESSURE MONITORING IN A MODULAR ADMINISTERING DEVICE

(76) Inventor: Rolf Marggi, Bern (CH)

(21) Appl. No.: 12/951,851

(22) Filed: Nov. 22, 2010

Related U.S. Application Data
(63) Continuation of application No. PCT/CH2009/000160, filed on May 15, 2009.

(30) Foreign Application Priority Data
May 23, 2008 (CH) CH 775/08

(51) Int. Cl. A61M 5/168; G01L 9/00; G01L 7/00

(52) U.S. Cl. 604/500; 604/67; 73/753; 73/714

(57) ABSTRACT

An administering device for administering a fluid product through the use of pressure, the device being modular, including a base unit and a cartridge, the base unit containing driving components and the cartridge adapted to be connected detachably to the base unit, wherein the cartridge has a fluid reservoir and a pressure monitoring device having a pressure sensor and a transfer device operably coupled to the pressure sensor, wherein the pressure monitoring device can be activated by an externally applied, alternating electromagnetic field, whereby data can be read, without contact, using the fluid pressure. In one embodiment, the pressure sensor contains a snap disk and the transfer device is an RFID transponder, wherein the base unit comprises a pressure reading device, which is constructed for producing a corresponding alternating electromagnetic field and, depending on the response to the alternating field, for determining a fluid pressure-dependent property of the transfer device.

### CROSS-REFERENCED RELATED APPLICATIONS

This application is a continuation of International Patent Application No. PCT/CH2009/000160 filed May 15, 2009, which claims priority to Swiss Patent Application No. 775/08 filed May 23, 2008, the entire contents of each of which are incorporated herein by reference.

### BACKGROUND

The present invention relates to devices for injecting, administering, infusing, delivering or dispensing a substance, and to methods of making and using such devices. More particularly, it relates to an administering device for administering a fluid product, e.g. a fluid medicament or therapeutic substance, the device having a modular design and comprising a reusable base unit (“reusable module”) with drive components and a cartridge (“disposable module”) that can be detachably connected to the base unit, the cartridge comprising a container for the fluid product. The present invention also relates to a base unit and a cartridge for such an administering device, and to a method for controlling such an administering device.

WO 2007/131367 discloses a modular administering device for a fluid medicament, which comprises a base unit and a cartridge that can be detachably connected thereto. An electrically operated drive apparatus is present in the base unit for generating a drive motion that is transferred to the cartridge. Moreover, there is a control apparatus in the base unit for controlling the drive apparatus and thus matching the administering rate to the individual requirements of a patient. Since these components are relatively expensive, the base unit is embodied as a reusable unit (“reusable module”). By contrast, the cartridge can be disposed of after a single use (“disposable module”). It contains a product container in the form of a carpule with a displaceable piston. Displacing the piston ejects the product from the product container. The product piston is displaced by hydraulic force transfer. To this end, the cartridge comprises a hydraulic reservoir with a hydraulic fluid, with the base unit being adapted to exert drive pressure thereon. A fluid connection extends from the hydraulic reservoir to a displacement reservoir, which is in part delimited by the product piston. When the drive pressure is exerted on the hydraulic reservoir, the former is transferred to the displacement reservoir via the fluid connection. As a result of this, a force is exerted on the product piston, which leads to the product piston being displaced and, as a result thereof, the product being ejected from the product container.

Such an administering device is used in particular for administering a medicament that is present in a liquid form, for example an insulin preparation or a blood-thinning medicament such as Heparin, to a patient over a relatively long period of time. In the process, it is essential that malfunctions, which could lead to an undersupply of the medicament or to a complete breakdown in the administration of the medicament, are recognized in an effective and reliable fashion. In particular, such a malfunction can occur as a result of an occlusion in a liquid-carrying line, for example in the infusion set or in the fluid connection of the hydraulic force transfer, or as a result of the piston in the product container jamming in such a way that the piston cannot be advanced any further due to insufficient drive pressure.

In the art, various measures have been proposed for identifying such occlusions. By way of example, U.S. Pat. No. 5,097,122 discloses an optical motion sensor for monitoring the drive motion of an administering device. U.S. Pat. No. 5,462,525 proposes an inductive through-flow measurement at the medicament outlet of the administering device. A pressure difference along a pressure-reducing capillary, through which the medicament to be administered passes, is monitored in WO 03/045302. The drive current of an electric motor drive in an administering device is monitored in US 2006/0184154. The reactive force, with which the piston in the medicament container counteracts its own advance, is measured and monitored in U.S. Pat. No. 5,647,853. In part, these methods are relatively complex and harbor the risk of errors occurring during the calibration or during the actual monitoring. Moreover, these methods in part rely on measuring variables that only allow indirect conclusions to be drawn in respect of an omitted administration of the medicament.

A further disadvantage of some of the known measures for identifying occlusions is that they cannot be used in an administering device with a modular design without further measures or adaptations. Thus, e.g. in the case of methods for identifying occlusions in which a through-flow rate is established directly or in which a force is measured, it is difficult to transfer the result of such a measurement from the disposable cartridge to the control or controller of the drive apparatus housed in the base unit in an efficient and, at the same time, cost-effective fashion.

### SUMMARY

According to a first aspect of the present invention, a cartridge for an administering device for administering a fluid product, e.g. a therapeutic substance, fluid medicament, etc., is provided, which allows malfunctions due to occlusions or other impediments to the administration to be identified and allows efficient, secure and cost-effective transfer of this information to a base unit interacting with the cartridge.

In one embodiment, the present invention comprises an administering device for administering a fluid product through the use of pressure, the device being modular, comprising a base unit and a cartridge, the base unit containing driving components and the cartridge adapted to be connected detachably to the base unit, wherein the cartridge comprises a fluid reservoir and a pressure monitoring device having a pressure sensor and a transfer device operably coupled to the pressure sensor, wherein the pressure monitoring device can be activated by an externally applied, alternating electromagnetic field, whereby data can be read, without contact, using or based on the fluid pressure. In one embodiment, the pressure sensor comprises a snap disk and the transfer device is an RFID transponder constructed for producing a corresponding alternating electromagnetic field and, depending on the response to the alternating field, for determining a fluid pressure-dependent property of the transfer device.

In one embodiment, the present invention comprises an administering device for administering a fluid product through the use of pressure, the device having a modular construction and comprising a base unit, which contains driving components, as well as a cartridge, which can be connected detachably to the base unit and has a fluid reservoir. The cartridge contains a pressure monitoring device, which has a pressure sensor as well as a transfer device, which is connected with the pressure sensor and can be activated by an externally applied, alternating electromagnetic field. In this way, relevant data can be read contactlessly from the fluid pressure from the transfer device. In an exemplary preferred embodiment, the pressure sensor contains a snap disk. In an exemplary preferred embodiment, the transfer device is an RFID transponder. Accordingly, in some preferred embodiments, the base unit comprises a pressure reading device, which is constructed for producing a corresponding alternating electromagnetic field and, depending on the response to the alternating field, for determining a fluid pressure-dependent property of the transfer device.

In one embodiment, a cartridge in accordance with the present invention comprises a fluid reservoir adapted to be subjected to a fluid pressure, and a pressure monitor interacting with the fluid reservoir. The product can be delivered from the cartridge as a result of pressure on the fluid reservoir. The pressure monitor comprises a pressure sensor and a transfer apparatus, which is connected to the pressure sensor. The transfer apparatus can be activated by an externally applied, alternating electromagnetic field and has at least one property that can be determined by the activation, the property being variable as a function of the fluid pressure established by the pressure sensor.

The present invention affords the possibility of identifying occlusions in an efficient and direct fashion by an increase in the fluid pressure in the fluid reservoir and transferring corresponding information to the base unit in a contactless fashion. As the property relating to the fluid pressure is read out by electromagnetic activation, disadvantages that would result from transfer via electrical contacts or a mechanical transfer are avoided. More particularly, such electromagnetic data transfer is not susceptible to contaminations, tolerances in the cartridge or base unit dimensions, or to tolerances in the precise arrangement of the components responsible for transfer in the cartridge and in the base unit. Moreover, in addition to pressure-dependent information, electromagnetic transfer offers the possibility of transferring further data from the cartridge to the base unit, or vice versa, for example data relating to the medicament contained in the cartridge, the dosage, the fill-level of the cartridge, operational conditions or states, times of earlier administrations, etc.

In a preferred embodiment, a transfer apparatus in accordance with the present invention comprises a transponder, which has at least one antenna for receiving an external alternating electromagnetic field, and an electronic receiver circuit connected to the antenna. Herein, in general terms, a transponder is understood to be an apparatus acting as a receiver for the alternating electromagnetic field and having a function consisting of automatically emitting signals when the externally generated sampling (activating) field is received. Thus, a transponder itself emits data after being activated. This can be brought about e.g. by active emission of a further electromagnetic field (e.g. at a different frequency) or, in some preferred embodiments, by modulating the activating field, e.g. by modulating the energy uptake by the transponder from the activating field (load modulation). In particular, the functional principle of such a transponder can be based on inductive coupling (in the near-field region) to the transmitter antenna of the activating field and, after activation, amplitude modulation of the activating field as a function of the data to be transferred, which modulation is brought about by time-varying inductive energy uptake from the activating field (AM-principle).

Transponders are widely used in the prior art. By way of example, they are used in anti-theft systems in stores, for identifying and tracing goods in transportation and automation, for marking animals, etc. The prior art distinguishes between passive and active transponders. Passive transponders are understood to be systems that obtain the energy required for their function from the activating field only. Passive transponders thus operate without their own permanent source of energy. By contrast, active systems have their own energy supply at their disposal, often in the form of a battery. In the context of some preferred embodiments of the present invention, a passive transponder is preferred to an active transponder for reasons of costs and for environmental reasons. In some embodiments, to ensure high data security, it may be advantageous in the present context to encrypt the transferred data.

Very different properties of a transponder, which can be influenced by the pressure sensor in a pressure-dependent fashion, can be considered for being determined in the case of activation by the external field. In some preferred embodiments, one or more data bits may be changed in the transponder as a function of pressure and may be emitted by the transponder after the activation, i.e. the pressure-dependent variable property of the transponder may be the state of at least one data bit in the transponder. In this case, the information relating to the fluid pressure is read out as the result of a transfer of the corresponding data bits by the transponder. In cases, only one data bit is dependent on the fluid pressure, wherein this bit is not set when a certain threshold pressure is undershot and is set when this threshold pressure is exceeded (or vice versa). However, there may also be a plurality of bits, wherein, for example, the binary number represented by these bits can represent a measure for the current fluid pressure. In this case, the transfer apparatus can have an analog/digital converter for converting an analog output signal from the pressure sensor into a binary value.

Many cost-effective, commercially available transponder chips do not have an external interface for setting data bits as a function of an external variable such as, for example, the output signal of a pressure sensor. In this case, data bits from the transponder cannot readily be changed as a function of the pressure. An alternative is that of changing a different property of the transponder as a function of the fluid pressure and establishing this property, e.g. an electrical property such as the sensitivity or impedance of the receiver circuit in the transponder. More particularly, it is proposed to short circuit or short the transponder antenna if a predetermined fluid pressure is exceeded, or to electrically disconnect said antenna from the receiver circuit, by the pressure sensor having an electrical contact connected in parallel to or in series with the antenna. In this case, an occlusion is identified by virtue of the fact that the transponder can only be read out as long as the fluid pressure is within normal parameters, i.e. below the threshold pressure. As soon as the base unit no longer receives a signal from the transponder, this indicates a malfunction, which triggers a corresponding alarm and/or further measures in the base unit.

Alternatively, the data relating to the fluid pressure can also be read out by virtue of the fact that the transponder has a resonant frequency that is dependent on the fluid pressure. By way of example, this can be achieved by virtue of the fact that the pressure sensor changes the value of a capacitance as a function of the pressure, wherein this capacitance is connected in parallel to or in series with the antenna of the transponder (which antenna generally acts predominantly as an inductor). In this case, data relating to the fluid pressure is read out by establishing the resonant frequency. In this case, it is also feasible for the transponder to be able to assume two possible resonant frequencies, wherein the first of the two resonant frequencies corresponds to a state in which a predetermined threshold pressure is undershot, whereas the second resonant frequency corresponds to a state in which this threshold pressure is exceeded.

To change a property of the transfer apparatus in one of these ways, the pressure sensor can accordingly have, for example, a fluid-pressure-dependent electrical resistance, a fluid-pressure-dependent capacitance, or a fluid-pressure-dependent inductance, or a fluid-pressure-dependent combination of these variables. As already explained in an exemplary fashion, the pressure sensor may have an electrical contact that assumes either an opened or a closed state as a function of the fluid pressure. More particularly, the pressure sensor can have a snap-action disk, which snaps from a stable first mechanical state into a metastable second mechanical state when a predetermined threshold pressure is exceeded. In the process, the snap-action disk changes an electrical property of the pressure sensor, e.g. the electrical resistance thereof. In one case, the snap-action disk opens or closes an electrical contact when snapping between two states. Such snap-action disks, which, when pressure is exerted thereon, snap from one defined position into another defined position, are well-known in the prior art. By way of example, they can be obtained from Inovan GmbH & Co. KG, Birkenfeld, Germany. An example of a particular snap-action disk is specified in EP 0 395 779 A2, but a differently designed snap-action disk may also be used. The snap-action disk can form a contact partner of the contact to be closed, wherein the second contact partner is arranged below the snap-action disk and separate therefrom, and so the snap-action disk only contacts the second contact partner in the second, metastable state. In this case, there needs to be an electrical supply line to the snap-action disk. However, the snap-action disk can also connect two contact partners formed independently from the disk by having an electrically conductive design, touching these two contact partners in its metastable second state and thus allowing a current to flow between said contact partners. However, finally it is also feasible for the snap-action disk to actuate a switch, e.g. a microswitch, when snapping between two states, which switch is insulated electrically from the snap-action disk. In this case, the snap-action disk need not be electrically conductive.

In one case, the fluid reservoir, whose pressure is monitored by the pressure sensor, and the product reservoir for the product to be administered are identical. However, in some preferred embodiments, the fluid reservoir, whose pressure is monitored, is used as a hydraulic reservoir, the pressure in which can be transferred onto the product reservoir by way of a hydraulic force transfer, to supply the fluid product from the product reservoir. This prevents the product to be administered from coming into direct contact with the pressure sensor and allows the product to be administered to be filled into a standardized container that need not be specifically adapted for pressure monitoring. Moreover, the position of the pressure sensor may be selected freely within broad limits and in accordance with the spatial conditions.

Another aspect of the present invention is a base unit for an administering device for administering or delivering a medicinal substance, e.g. a fluid therapeutic product. Such a base unit may comprise a pressure readout apparatus designed to generate an alternating electromagnetic field and to establish a property relating to the fluid pressure in a fluid reservoir of a cartridge as a function of a response to the alternating field. In general, the base unit additionally comprises a drive apparatus for generating a drive motion to exert the fluid pressure on the fluid reservoir in the cartridge. Additionally, the base unit generally comprises a control apparatus designed to control the drive apparatus. The control apparatus can then be designed such that during operation it controls the drive apparatus as a function of the property established by the pressure readout apparatus. Alternatively, or in addition thereto, there can be an output apparatus, which is designed to output optical, acoustic and/or tactile information during operation as a function of the property established by the pressure readout apparatus. By way of example, this can be a display, a luminous element (small lamp, LED, etc.), a buzzer, a vibration alarm, etc. The emitted information or signals can comprise signals for, e.g. normal operation, an occlusion warning after exceeding a threshold pressure for the first time, an occlusion alarm after repeatedly exceeding the threshold pressure, etc.

In some preferred embodiments, the pressure readout apparatus comprises a transceiver, which has at least one antenna for emitting an alternating electromagnetic field, and an electronic transmitter/receiver circuit connected to the antenna. In general terms, the term transceiver is understood to mean an apparatus acting both as a transmitter and as a receiver and the function of which at least comprising the act of generating a sampling (activating) field and receiving signals emitted by a transponder as a response thereto. Such transceivers are widespread in the prior art and are used wherever transponders are intended to be read out. More particularly, the transceiver can be designed to establish a time-dependent modulation of the amplitude of the activating field by an alternating inductive load.

According to a further aspect of the present invention, an administering device is provided for administering a fluid product, which administering device comprises a cartridge of the type described above and a base unit of the type described above that can be detachably coupled.

In some embodiments, the present invention comprises a method for controlling an administering device for administering a fluid product. In some embodiments, such a method comprises the following steps:
generating an alternating electromagnetic field in a base unit of an administering device;
activating a pressure monitor, contained in a cartridge of the administering device, by the alternating electromagnetic field;
establishing at least one property of the pressure monitor by the base unit as a result of a response of the pressure monitor to the alternating electromagnetic field.

As already explained above, in some embodiments, provision can be made for an optical, acoustic or tactile output apparatus, e.g. a display, a luminous element, a buzzer, a vibration alarm, etc., to be actuated as a function of the established property, e.g. in the case where a predetermined fluid pressure is exceeded for the first time or exceeded repeatedly. Alternatively, or in addition thereto, provision can be made for a drive apparatus, contained in a base unit, to be controlled as a function of an established property of a pressure monitor, for example said drive apparatus is stopped, or operated in the opposite direction, when a predetermined fluid pressure is exceeded for the first time or exceeded repeatedly.

### BRIEF DESCRIPTION OF THE DRAWINGS

FIG. 1 is a perspective sectional view of an embodiment of an administering device according to the present invention;

FIG. 2 is a longitudinal section through the administering device of FIG. 1;

FIG. 3 is a schematic perspective view of a pressure monitor and a pressure readout apparatus interacting therewith;

FIG. 4 is a schematic perspective view of only the pressure monitor of FIG. 3;

FIG. 5 is a side view of the pressure monitor of FIG. 4;

FIG. 6 is a top view of the pressure monitor of FIG. 4; and

FIG. 7 is a front view of the pressure monitor of FIG. 4.

### DETAILED DESCRIPTION

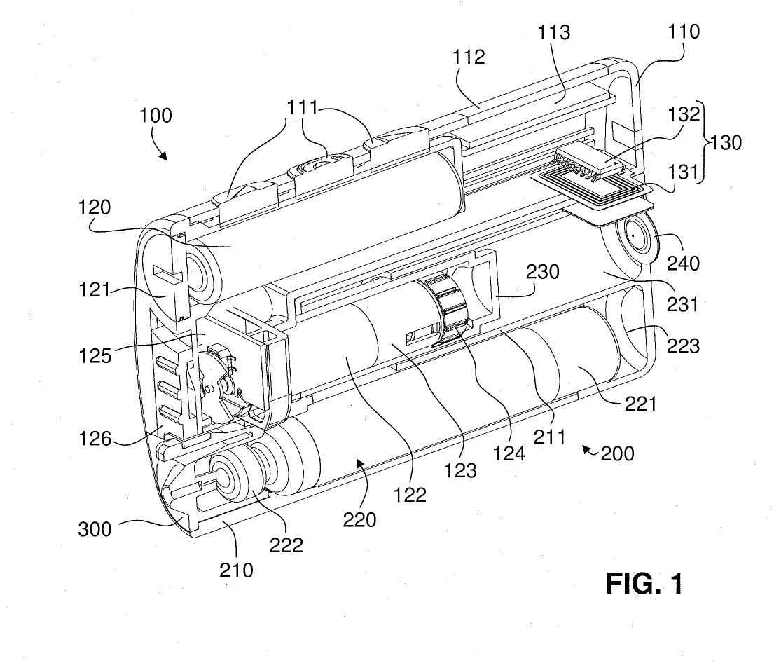

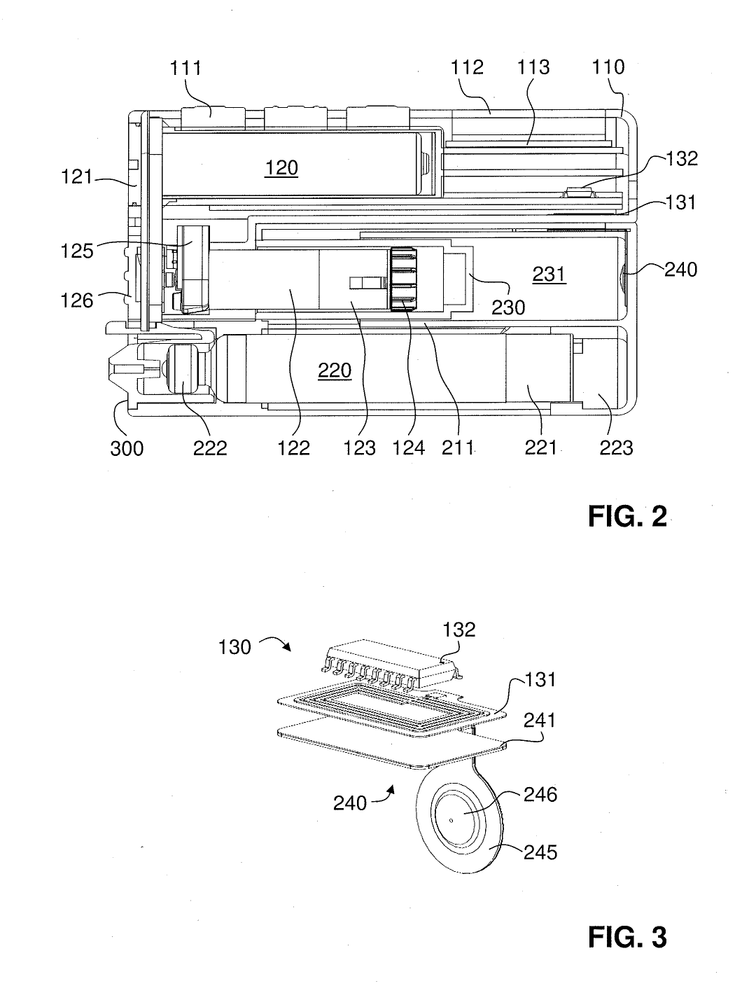

FIGS. 1 and 2 schematically illustrate a modular administering device for administering a liquid medicament. The device comprises a reusable base unit 100 and a disposable cartridge 200 that complements the base unit.

With regard to fastening, mounting, attaching or connecting components of the present invention, unless specifically described as otherwise, conventional mechanical fasteners and methods may be used. Other appropriate fastening or attachment methods include adhesives, welding and soldering, the latter particularly with regard to the electrical system of the present invention, if any. In embodiments with electrical features or components, suitable electrical components and circuitry, wires, wireless components, chips, boards, microprocessors, inputs, outputs, displays, control components, etc. may be used. Generally, unless otherwise indicated, the materials for making embodiments of the present invention and/or components thereof may be selected from appropriate materials such as metal, metallic alloys, ceramics, plastics, etc. Unless otherwise indicated specifically or by context, positional terms (e.g., up, down, front, rear, distal, proximal, etc.) are descriptive not limiting. Same reference numbers are used to denote same parts or components.

Direction designations for specifying directions within the administering device are defined as follows. The distal direction should be understood to mean that direction in which a relevant moveable element of the administering device moves while the medicament is being administered. As will be explained in more detail below, a direction of advance is deflected by 180° in the interior of the administering device. Hence the distal direction corresponds to different absolute spatial directions in different parts of the administering device. The proximal direction is defined in each case as the direction opposite to the distal direction. A lateral direction is a direction perpendicular thereto.

The cartridge 200 comprises a housing 210 in which a product container 220 in the form of a carpule (which also may be thought of and/or referred to as a vial, ampoule, container, reservoir, etc.) with a cylindrical sidewall region and a product piston 221 that can be displaced therein is housed in the region illustrated toward the bottom in FIG. 1. At its distal end, the product container 220 is sealed by a closure cap 222, which has only been illustrated schematically and has a septum, and it thus bounds a product reservoir (which also may be referred to and thought of as a medicament reservoir). In the region of the cartridge illustrated toward the top in FIG. 1 there is a hydraulic reservoir 231, which is delimited in the lateral direction by a cylindrical sidewall region 211 of the housing 210. In the proximal direction, the hydraulic reservoir is delimited by a hydraulic piston 230, which can be moved in the axial direction with respect to the housing and is guided in a sealing fashion. A fluid channel not illustrated in FIG. 1 is used to connect the hydraulic reservoir 231 to a displacement reservoir 223, which is arranged bottom right in FIG. 1 and delimited in the distal direction by the product piston 221. A hydraulic fluid, for example colored, deionized water or a suitable oil, has been filled into the hydraulic reservoir 231, into the displacement reservoir 223 and into the fluid channel.

On its outer side, the hydraulic piston 230 has a male thread, which is not illustrated in any more detail in FIG. 1 and engages with a corresponding female thread formed on the inner side of the sidewall region 211 delimiting the hydraulic reservoir. The hydraulic piston has a hollow interior and so overall it forms an elongate, cylindrical, hollow-spindle-like sleeve. A multiplicity of longitudinal ribs (not illustrated in FIG. 1) are formed in the interior of this sleeve and run or extend parallel to the longitudinal direction of the sleeve.

The base unit 100 has a housing 110, which houses a battery 120 or rechargeable battery that can be accessed via a battery compartment lid 121, a drive motor 122 arranged on a base board 125, a transmission 123, and various components used for controlling the drive motor, illustrated in part. A plurality of operating buttons 111 (three in the present example) and a display 113 visible through a window 112 are arranged on the top side of the base unit. The control apparatus of the base unit 100 can be programmed with respect to the individual requirements of the patient using these operating elements.

The motor 122 drives a drive 124 to a driving rotary motion via the transmission 123. The drive 124 consists essentially of a wheel, with a multiplicity of drive ribs extending in the axial direction being arranged on its circumferential surface. A base unit with, in principle, a similar design is described in the international application PCT/CH2007/000113, dated Mar. 2, 2007; reference is made thereto and incorporated herein in respect of further details of one suitable design of the base unit and the force transfer between base unit and cartridge. (See also US Publication 2010152661, owned by the owner of the present application.)

To administer the medicament in the product container 220, the cartridge 200 is at first connected to the base unit 100, as illustrated in FIG. 1. In this state, the motor, the transmission and the drive form a finger-like structure that projects into the interior of the hydraulic piston 230. The drive ribs of the drive engage into the interspaces between respectively two longitudinal ribs of the hydraulic piston and thereby connect the drive 124 to the hydraulic piston 230 in a way that is rotationally fixed but can be displaced in the longitudinal direction.

A needle adaptor 300 is thereupon placed onto the cartridge, with a catheter of an infusion set (not illustrated in FIG. 1) adjoining the former. The needle adaptor 300 comprises a hollow needle that pierces the septum of the closure cap 222 and thus connects the interior of the product container 220 to the catheter. The cartridge 200 and the needle adaptor 300 are fixed to the base unit 100 by a displaceable bolt 126.

After priming the catheter, a certain amount of product is dispensed at predefined intervals (e.g. three times an hour) during normal operation. To this end, the motor 122 sets the drive 124 into rotary motion via the transmission 123. This rotary motion is transferred to the hydraulic piston 230 because the drive 124 engages with the longitudinal ribs on the inner side of said hydraulic piston. Since the hydraulic piston 230 is guided in the housing 210 of the cartridge 200 by a threaded engagement, the rotary motion of the hydraulic piston 230 at the same time advances the hydraulic piston in the distal direction. Thus, overall, the hydraulic piston 230 undergoes a helical motion in the distal direction. This reduces the volume in the hydraulic reservoir 231, and so the hydraulic fluid is pressed into the displacement reservoir 223 through the fluid channel and here this leads to an advance of the product piston 221 in the distal direction. This finally ejects the fluid product out of the now likewise reducing interior of the product container 220 through the hollow needle and the catheter. Thus, rotating the hydraulic piston 230 ultimately results in an advance thereof and in this way an ejection of the fluid product from the product container 220 via the hydraulic arrangement.

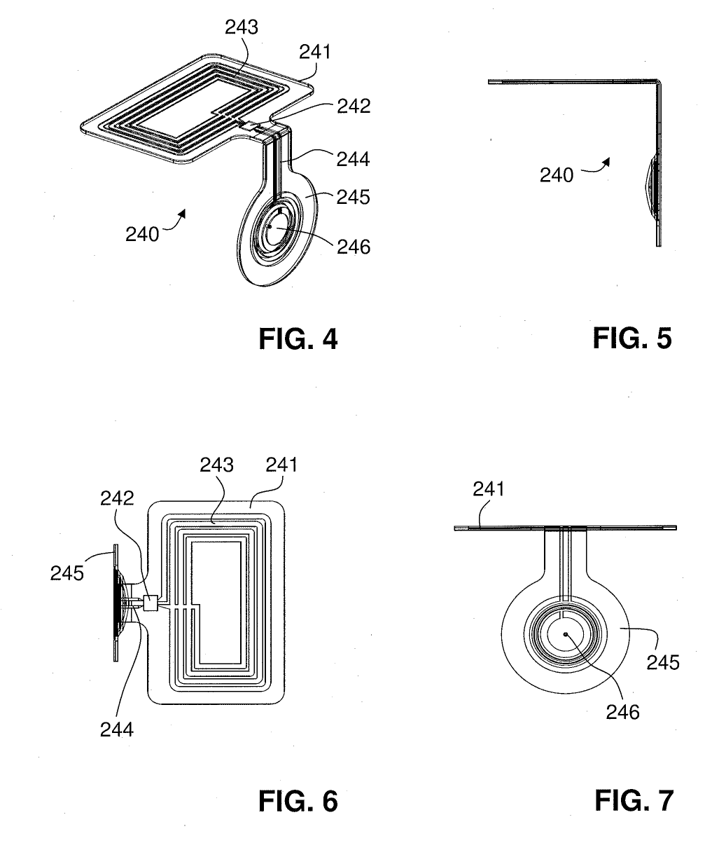

To be able to identify occlusions, the cartridge 200 comprises a pressure monitor 240, which is illustrated in more detail in FIGS. 3 to 7. The pressure monitor 240 has a pressure sensor 245. In the present example, this pressure sensor is arranged at the distal end of the hydraulic reservoir 231 in a distal end face region of the latter. It delimits the hydraulic reservoir in the distal direction. However, the pressure sensor can also be attached anywhere else in the region that is filled by the hydraulic fluid. In the present example, the pressure sensor has a plastics support that bears a snap-action disk 246. This snap-action disk can assume two positions with respect to the axial direction. As long as pressure does not load the snap-action disk, or as long as the pressure on the snap-action disk does not exceed a certain threshold pressure, the snap-action disk is in a first, stable position. However, if the pressure in the hydraulic reservoir exceeds the threshold pressure, the snap-action disk jumps into a metastable, second defined position in which it is displaced in the distal direction by a certain, relatively small amount compared to the first position. The transition between these two positions is a sudden snapping-like motion. Such snap-action disks are well-known in a different context in the prior art and are used e.g. in keyboards.

When the snap-action disk snaps into the second position, it closes an electrical contact in the process, as is also already known per se from the prior art. For this purpose, the snap-action disk itself can be made of a metal in particular or otherwise carry an electrically conductive contact site. Additionally, there preferably is an electrically conductive counter element adjacent to the snap-action disk in the distal direction, onto which counter element the snap-action disk, or the contact site attached thereon, impacts when snapping between the two states in order thus to establish an electrical connection between the snap-action disk and counter element. Alternatively, there can also be two electrically conductive counter elements, which can be interconnected electrically by the contact site on the snap-action disk. This removes the need for an electrical supply line to the (moveable) snap-action disk.

The electrical contact formed thus is connected to a transfer apparatus 241 in the form of a radio-frequency identification (RFID) transponder via two connection lines 244. The transponder has a flat-coil antenna 243, mainly acting as an inductor, and a receiver circuit (transponder chip) 242 that can be identified particularly well in FIG. 3. Such transponders have been known for a long time per se.

The antenna 243 and the circuit 242 are housed on a common support, laminated in and thus protected from environmental effects, as is well-known per se in the prior art.

In the present exemplary embodiment, the electrical contact of the pressure sensor 245 is connected in parallel to the antenna 243. When the threshold pressure in the hydraulic reservoir is exceeded, and the snap-action disk 246 is therefore in its second position, this contact is closed and the antenna 243 is bridged (shorted) by this contact. This practically disables the transponder 241. However, as long as the threshold pressure has not been exceeded, the pressure sensor 245 contact is open and the transponder 241 can fulfill its normal function.

The support for the snap-action disk can be produced on the basis of plastics. More particularly, it is feasible that the pressure sensor and the transfer apparatus (transponder) are produced together in an integral fashion on a single support and that they are laminated together in a single plastics film. This protects the pressure monitor to a large extent from moisture and corrosion.

To communicate with the transfer apparatus 241 of the cartridge 200, a pressure readout apparatus 130 in the form of an RFID transceiver is housed in the base unit 100. The transceiver likewise has a flat-coil antenna 131 and a transceiver transmission/readout circuit (transceiver chip) 132. Such transceivers are also well-known per se in the prior art. By way of example, the transceiver chip EM4094 or EM4095 from EM Microelectronic in conjunction with a transponder compatible therewith is a suitable type of transceiver. The operating frequency of the transceiver/transponder pair can be any usual operating frequency for such readout apparatuses. In particular, known frequency bands are the regions around 125 kHz and 13.56 MHz, but other frequencies are also possible.

The flat antenna coils of the readout apparatus 130 and the transfer apparatus 241 are arranged at least approximately parallel and opposing one another in regions in the vicinity of the respective external wall of the base unit or the cartridge. The central coil axes of the antenna coils are parallel to one another and preferably coincide. This opposing, parallel arrangement of the coils ensures optimum inductive coupling of the coils and hence a high signal strength.

During normal operation, the transceiver is actuated by a control apparatus (not illustrated in any more detail) in the base unit such that the antenna 131 of the transceiver generates a sampling electromagnetic field at regular intervals, for example every time the motor 122 is put into operation. This electromagnetic field is received by the antenna 131 of the transponder in the cartridge and in turn causes the transponder itself to emit a certain data sequence in turn as a response thereto, for example by modulating the amplitude of the sampling field. By way of example, this data can comprise identification of the cartridge or further data relating to the cartridge, which will be explained in more detail below. The readout apparatus 130 receives this data using its antenna 131 and relays corresponding information to the control apparatus of the base unit.

However, if there is an occlusion in the cartridge 200 or in the infusion set adjoined thereto, the pressure in the hydraulic reservoir 231 will increase significantly if the hydraulic piston continues to advance. As soon as the threshold pressure has been exceeded, the snap-action disk 246 snaps into its second position and closes the contact of pressure sensor 245. This effectively renders the transponder of the transfer apparatus 241 useless. When the base unit next attempts to read out the transponder using the readout apparatus 130, the readout apparatus will not receive a signal from the transponder and therefore emit an error message to the control apparatus of the base unit 100. This emits an alarm signal for the user e.g. via the display 113 and/or a buzzer (not illustrated).

The proposed method for identifying occlusions allows an occlusion in the cartridge 200 or in the infusion set to be identified in efficient and cost-effective fashion, and corresponding information to be transferred contactlessly (i.e. without direct physical connection or contact) to the base unit. It offers at least the following advantages:
No openings are required in the cartridge because all data is transferred contactlessly. This reduces the risk of a leakage through which hydraulic fluid could escape.
Nor are openings required in the base unit, as would be required in e.g. mechanical occlusion detection.
Moving parts are dispensed with entirely.
The proposed arrangement is forgiving of tolerances in the dimensions of the base unit and the cartridge, and also toward the precise position of the transceiver and the transponder.
The assembly is more efficient than a mechanical occlusion identification, and the risk of assembly errors is reduced.
The hydraulic reservoir can have an entirely cylindrical basic shape without the need for eccentric sharpenings for a mechanical or another type of occlusion detection. This can help ensure the roundness and tightness of the hydraulic reservoir efficiently.
There is only a very small blockage bolus in the case of an occlusion because a response of the snap-action disk results in a very small change of volume in the hydraulic reservoir.
The response behavior is very precise and repeatable.

Alternatively, it is feasible for the antenna of the transponder not to be shorted by the contact but for the resonant frequency of the receiver resonant circuit in the transponder to be changed by closing the contact. In this case an occlusion is identified by determining the resonant frequency.

In a further alternative preferred embodiment, at least one data bit of the transponder 241 can be changed directly by an external contact. In this case, the contact of the pressure sensor is no longer needed to short the antenna, but is used to change the corresponding data bit in the transponder. In contrast to the method described above, the transponder thus remains fully functional, even in the case of an occlusion. The readout apparatus will now read out the corresponding data bit of the transponder and will use this bit to determine whether or not there is an occlusion.

A multiplicity of further options for transferring the information in respect of the fluid pressure are possible. Thus, it is also feasible to use a continuously acting pressure sensor instead of a snap-action disk, the former continuously changing its electrical properties as a function of pressure. By way of example, this can be brought about in a resistive, capacitive or inductive fashion. Accordingly, it is feasible to continuously change the resonant frequency of the transponder as a function of the fluid pressure, or it is feasible to convert the pressure recorded by the pressure sensor into a digital value using an analog/digital converter (ADC) and to store said value in the transponder and to read out this value in the case of a query by the readout apparatus.

Furthermore, additional data can be stored in the transponder chip in the transfer apparatus 241. More particularly, the current fill level of the cartridge can be stored. In this case the fill level can be determined e.g. by a direct measurement, for example by optical means, or it can be established indirectly, e.g. by the number of rotations completed by the drive motor 122.

To store such data, the transponder comprises a memory interacting with the receiver circuit, which memory has a multiplicity of data bits for storing and outputting data. This memory can be, subdivided into various regions, wherein individual regions can be read only, whereas other memory regions can be changed, for example by externally received information or by an electrical interface.

By way of example, each cartridge can be assigned an unambiguous sequence of digits in the form of the serial number of the transponder chip, which is stored in a read-only region in the memory. As a result, each individual cartridge can be identified during production, distribution, use and in the case of problems with the quality.

Further data that could be stored and changed where necessary comprises, for example, an identification of the contained medicament, the date of filling (to avoid too long a storage), and data relating to temperatures that the cartridge was subjected to during storage or during operation.

By way of example, the useful data saved in the transponder can be stored in the following form:

Serial number (32 bit)-Level (16 bit)-Occlusion (1 bit).

Whereas an administering device with hydraulic pressure transfer was described above, the invention can also be used in administering devices without a hydraulic force transfer. In this case the pressure sensor directly measures the pressure in the product container.

Embodiments of the present invention, including preferred embodiments, have been presented for the purpose of illustration and description. They are not intended to be exhaustive or to limit the invention to the precise forms and steps disclosed. The embodiments were chosen and described to illustrate the principles of the present invention and the practical application thereof, and to enable one of ordinary skill in the art to utilize the invention in various embodiments and with various modifications as are suited to the particular use contemplated. All such modifications and variations are within the scope of the invention as determined by the appended claims when interpreted in accordance with the breadth they are fairly, legally, and equitably entitled.

1. A cartridge for an administering device for administering a fluid medicament by applying pressure, comprising:
a fluid reservoir adapted to be subjected to a fluid pressure, and
a pressure monitor interacting with the fluid reservoir, the pressure monitor comprising:
a pressure sensor, and
a transfer apparatus operably coupled to the pressure sensor and adapted to be activated by an externally applied, alternating electromagnetic field to allow a contactless readout of data from the transfer apparatus,
wherein the transfer apparatus has at least one property to be determined by the activation, the property varying as a function of the fluid pressure sensed by the pressure sensor.

2. The cartridge as claimed in claim 1, wherein the transfer apparatus comprises a transponder having at least one antenna for receiving the external alternating electromagnetic field and an electronic receiver circuit, wherein the transponder is adapted to be activated by the externally applied alternating electromagnetic field.

3. The cartridge as claimed in claim 2, wherein the transponder modulates the external alternating electromagnetic field to allow the readout of data.

4. The cartridge as claimed in claim 2, wherein the pressure sensor is connected to the transponder such that the transponder antenna is shorted when a predetermined fluid pressure is exceeded.

5. The cartridge as claimed in claim 2, wherein the transponder has a resonant frequency which depends on the fluid pressure.

6. The cartridge as claimed in claim 2, wherein the transponder has, interacting with the transmitter/receiver circuit, a memory region with a plurality of data bits for storing and outputting data, and wherein at least one of the data bits is adapted to be changed as a function of the fluid pressure.

7. The cartridge as claimed in claim 1, wherein the pressure sensor has at least one of a fluid-pressure-dependent electrical resistance, a fluid-pressure-dependent capacitance and a fluid-pressure-dependent inductance.

8. The cartridge as claimed in claim 1, wherein the pressure sensor comprises an electrical contact that assumes either an opened or a closed state as a function of the fluid pressure.

9. The cartridge as claimed in claim 1, wherein the pressure sensor comprises a snap-action disk, which snaps from a first mechanical state into a second mechanical state when a predetermined threshold pressure is exceeded and thus changes an electrical property of the pressure sensor.

10. The cartridge as claimed in claim 1, further comprising a medicament reservoir for the fluid medicament, the medicament reservoir formed separately from the fluid reservoir, wherein the fluid pressure in the fluid reservoir can be transferred to the medicament reservoir to deliver the medicament from the medicament reservoir.

11. A base unit for an administering device for administering a fluid medicament using pressure, the base unit designed to interact with a cartridge comprising a medicament reservoir, the base unit comprising a pressure readout apparatus designed to generate an alternating electromagnetic field and to establish a property relating to a fluid pressure in the medicament reservoir of the cartridge as a function of a response to the electromagnetic field.

12. The base unit as claimed in claim 11, wherein the pressure readout apparatus comprises a transceiver and an electronic transmitter circuit, the transceiver comprising at least one antenna for emitting the alternating electromagnetic field and for receiving data as a response to the activating alternating field, the electronic transmitter circuit operably coupled to the antenna.

13. A base unit for an administering device for administering a fluid medicament using pressure, the base unit designed to interact with a cartridge comprising a medicament reservoir, a fluid reservoir adapted to be subject to pressure and a pressure monitor interacting with the fluid reservoir, the pressure monitor comprising a pressure sensor and a transfer apparatus operably coupled to the pressure sensor and adapted to be activated by an externally applied alternating electromagnetic field to allow a contactless readout of date from the transfer apparatus, wherein the transfer apparatus has at least one property determined by the activation, the property varying as a function of the fluid pressure sensed by the pressure sensor, the base unit comprising a pressure readout apparatus designed to generate the alternating electromagnetic field and to establish the at least one property as a function of a response to the electromagnetic field.

14. The base unit as claimed in claim 13, wherein the pressure readout apparatus comprises a transceiver and an electronic transmitter circuit, the transceiver comprising at least one antenna for emitting the alternating electromagnetic field and for receiving data as a response to the activating alternating field, the electronic transmitter circuit operably coupled to the antenna.

15. An administering device for administering a fluid product, comprising a cartridge a cartridge comprising a medicament reservoir, a fluid reservoir adapted to be subject to pressure and a pressure monitor interacting with the fluid reservoir, the pressure monitor comprising a pressure sensor and a transfer apparatus operably coupled to the pressure sensor and adapted to be activated by an externally applied alternating electromagnetic field to allow a contactless readout of date from the transfer apparatus, wherein the transfer apparatus has at least one property determined by the activation, the property varying as a function of the fluid pressure sensed by the pressure sensor, and a base unit designed to be detachably connected to the cartridge, the base unit comprising a pressure readout apparatus designed to generate the alternating electromagnetic field and to establish a property relating to the fluid pressure in the medicament reservoir of the cartridge as a function of a response to the electromagnetic field.

16. The administering device as claimed in claim 15, wherein the transfer apparatus comprises a transponder having at least one antenna for receiving the external alternating electromagnetic field and an electronic receiver circuit, wherein the transponder is adapted to be activated by the electromagnetic field.

17. The administering device as claimed in claim 16, wherein the pressure readout apparatus comprises a transceiver and an electronic transmitter circuit, the transceiver comprising at least one antenna for emitting the electromagnetic field and for receiving data as a response to the activating field, the electronic transmitter circuit operably coupled to the antenna.

18. A method for determining an operational state of an administering device for administering a fluid product, wherein the administering device comprises a cartridge having a fluid reservoir and a pressure monitor and a base unit that can be detachably connected to the cartridge, the method comprising the following steps:
generating an alternating electromagnetic field in the base unit;
activating the pressure monitor in the cartridge by the alternating electromagnetic field; and
establishing at least one property of the pressure monitor by the base unit as a result of a response of the pressure monitor to the alternating electromagnetic field.

19. The method as claimed in claim 18, further comprising the step of outputting optical and/or acoustic information as a function of the established property of the pressure monitor.

20. The method as claimed in claim 19, wherein the base unit comprises a drive apparatus for exerting a fluid pressure on the fluid reservoir, and wherein the method further comprises the step of controlling the drive apparatus as a function of the established property of the pressure monitor.
